(19) United States
(12) Patent Application Publication
(10) Pub. No.: US 2019/0040172 A1
(43) Pub. Date: Feb. 7, 2019

(54) AQUEOUS EMULSION POLYMERS AS RHEOLOGY MODIFIERS, COMPOSITIONS THEREOF, AND METHOD OF USE

(71) Applicant: ISP Investments LLC, Wilmington, DE (US)

(72) Inventors: Karen MITCHINSON, West Yorkshire (GB); David PETTY, Bradford (GB); Martin Swanson VETHAMUTHU, Stewartsville, NJ (US); Edward DiANTONIO, Staten Island, NY (US)

(73) Assignee: ISP Investments LLC, Wilmington, DE (US)

(21) Appl. No.: 16/074,993

(22) PCT Filed: Feb. 6, 2017
(86) PCT No.: PCT/US17/16632

§ 371 Date: Aug. 2, 2018

Related U.S. Application Data
(60) Provisional application No. 62/291,122, filed on Feb. 4, 2016.

(51) Int. Cl. C08F 220/06; C08F 220/18; C08F 220/14; C08K 5/14; C08K 5/23; C08K 3/30; C08F 2/26; C08F 2/30; A61K 8/04; A61K 8/81; A61Q 19/10

(52) U.S. Cl. CPC C08F 220/06; A61K 2800/21; C08F 220/14; C08K 5/14; C08K 5/23; C08K 3/30; C08F 2/26; C08F 2/30; A61K 8/042; A61K 8/8147; A61Q 19/10; C08K 2003/3054; C08K 2003/3072; C08F 2220/1825; C08F 2800/10; C08F 220/18

(57) ABSTRACT

The present application provides an aqueous emulsion polymer free of ethyl acrylate comprising (a) monomers (i) methyl acrylate, (ii) butyl acrylate and (iii) methacrylic acid; (b) crosslinker and (c) at least one surfactant. The present invention also provides the use of such emulsion polymers along with acid, cationic polymer, oil and other additives as low pH rheology modifiers and suspending aids for surfactant based micelle technology, emulsions and complex fluids in personal care, home care and industrial applications.

### FIELD OF THE INVENTION

The present application relates to aqueous emulsion polymers and more particularly, to emulsion polymers as low pH rheology modifiers and suspending aids for surfactant based micelle technology, emulsions and complex fluids in personal care, home care and industrial applications.

### BACKGROUND OF THE INVENTION

One particular purpose of providing distinctive structure to personal care, home care and industrial care products is to provide specific flow behavior. Specific types of applications often require specific flow behavior. Two structuring properties are often desired in personal care, home care or industrial care products: shear thinning capabilities and bead and/or particle suspension capabilities. The capability to suspend particles in principle is characterized by the yield stress value. High zero-shear viscosity values can also be indicative of particle suspension capability. Shear thinning capabilities are typically characterized by the pouring viscosity and the ratio of the pouring viscosity and low-stress viscosity values.

Conventional approaches for providing distinctive structure to personal care products include the addition of specific structuring agents, including both internal and external structuring agents. Examples of known internal structuring agents include surfactants and electrolytes. External structuring agents include polymers or gums, many of which are known to swell or expand when hydrated to form random dispersions of independent microgel particles.

Existing technologies exhibit insufficient yield and clarity under low pH conditions which can manifest itself as poor stability under different conditions e.g., elevated temperature. The formulator of low pH compositions, especially emulsions, has a limited choice of either nonionic thickeners, such as nonionic surfactants, or cationic thickeners.

U.S. Pat. No. 9,127,102 assigned to Conopco, Inc., discloses a rheology modifier copolymer comprising macro monomer, acrylic or methacrylic acid or salt thereof, ester of acrylic acid or methacrylic acid and an associative monomer.

U.S. Publication Number 20140341957 assigned to Lubrizol Advanced Materials Inc. discloses a crosslinked, nonionic amphiphilic polymer capable of forming a yield stress fluid in the presence of a surfactant.

Surfactant-Activated Microgels: A New Pathway to Rheology Modification (Krishnan Chari, et al., Langmuir 2013, 29, 15521-15528) discloses nonionic microgels composed of hydrophobic alkyl acrylates and hydrophilic hydroxyalkyl esters that utilize the effects of surfactant mediated swelling and interaction to provide pH independent rheological properties.

Ethyl acrylate is used as key monomer in the production of polymers along with other polymeric and has varied industrial applications. However, ethyl acrylate is labeled as “category 2B: possible components le carcinogen to humans” by International Agency for Research on Cancer (IARC). Manufacturers are in need for an alternative substitute retaining the same properties or uses.

In view of the foregoing, there exists an unmet need in the art for an aqueous emulsion polymer substantially free of ethyl acrylate and having thickening and suspension properties to surfactant systems at low pH.

The applicants have surprisingly found a specific aqueous emulsion polymer substantially free of ethyl acrylate that thickens at a low pH. Accordingly, the present application demonstrates aqueous emulsion polymers substantially free of ethyl acrylate form isotropically clear or opaque microgels as needed in aqueous surfactant media primarily used to suspend all types of beads, chemical peels, air bubbles, oil droplets, and exfoliating agents in personal care, home care and industrial applications.

### SUMMARY OF THE INVENTION

The primary objective of the present application is to provide aqueous emulsion polymers as low pH rheology modifiers and suspending aids for all types of surfactant based micelle technology, emulsions and complex fluids in personal care, home care and industrial applications.

In one important aspect, the present application provides an aqueous emulsion polymer comprising: (a) monomers: (i) from about 12 mole % to about 27 mole % of butyl acrylate; (ii) from about 23 mole % to about 50 mole % of methyl acrylate; and (iii) from about 30 mole % to about 50 mole % of methacrylic acid; (b) from about 200 ppm to about 3000 ppm of at least one crosslinking agent; and (c) from about 0.5 wt. % to 2.0 wt. % of at least one non-ionic or anionic surfactant. In preferred embodiments, the aqueous emulsion polymer consists essentially of, or consists of such monomers.

Ethyl acrylate is present in an amount not more than [NWT] 1.0 mole % of the total polymer composition. In preferred embodiments, the polymer composition is free of ethyl acrylate.

In yet another aspect, the present application provides an aqueous emulsion polymer comprising (a) monomers: (i) about 20 to about 25 mole % of butyl acrylate, (ii) about 40 to about 45 mole % of methyl acrylate, (iii) about 30 to about 40 mole % of methacrylic acid, (b) about 1000 ppm of at least one crosslinking agent; and (c) at least one surfactant which represented by the following structure:

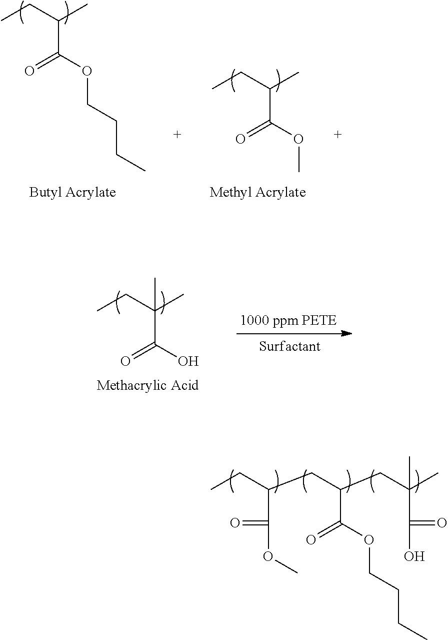

In another aspect, the present application provides an aqueous microgel composition comprising: (i) an aqueous emulsion polymer of: (a) monomers: (i) from about 12 mole % to about 27 mole % of butyl acrylate; (ii) from about 23 mole % to about 50 mole % of methyl acrylate; and (iii) from about 30 mole % to about 50 mole % of methacrylic acid; (b) from about 200 ppm to about 3000 ppm of at least one cross-linking agent; and (c) from about 0.5 wt % to 2.0 wt % of at least one anionic or non-ionic surfactant; (ii) at least one anionic, cationic, non-ionic or amphoteric surfactant, mixtures thereof; and (iii) optionally, at least one acid, at least one cationic polymer, at least one oil alone or combinations thereof.

The present application provides a microgel which thickens or swells in the presence of at least one surfactant directly at pH below 7, particularly between pH 3.5 to 6.5 through direct pH adjustment.

In still another aspect, the present application provides use of the microgel as a low pH rheology modifier and suspending aid for all types of surfactant based micelle technology, emulsions and complex fluids in stable personal care, home care and industrial care applications.

In yet another aspect, the present application provides an aqueous personal care microgel composition comprising: (i) an aqueous emulsion of: (a) monomers: (i) from about 12 mole % to about 27 mole % of butyl acrylate; (ii) from about 23 mole % to about 50 mole % of methyl acrylate; and (iii) from about 30 mole % to about 50 mole % of methacrylic acid; (b) from about 200 ppm to about 3000 ppm of at least one cross-linking agent; and (c) from about 0.5 wt. % to about 2.0 wt. % of at least one anionic or non-ionic surfactant; (ii) at least one anionic, cationic, non-ionic or amphoteric surfactant, mixtures thereof; (iii) at least one personal care additive; and (iv) optionally, at least one acid, at least one cationic polymer, at least one oil alone or combinations thereof.

In yet another aspect, the present application provides a process for preparing an aqueous emulsion polymer, wherein the process comprises: (a) preparing a pre-emulsion by mixing solutions of monomers: (i) from about 12 mole % to about 27 mole % of butyl acrylate; (ii) from about 23 mole % to about 50 mole % of methyl acrylate; and (iii) about 30 mole % to about 50 mole % of methacrylic acid; about 200 ppm to about 3000 ppm of at least one cross-linking agent; and about 0.5 wt. % to about 2.0 wt. % of at least one anionic or non-ionic surfactant in the dispersion medium; (b) adding polymerisation initiator and monomer pre-emulsion of step (a) to a reactor; (c) polymerizing reactants of step (b) at 75-80° C. for about 3-6 hours; and (d) cooling the reaction solution to obtain the aqueous emulsion polymer.

### BRIEF DESCRIPTION OF THE FIGURES

Further embodiments of the present application can be readily understood with reference to the appended figures.

FIG. 1 illustrates rheology and bead test in microgel composition kept at pH 4.5, 5.5 and 6.5 for 3 months at 45° C.

FIG. 2 illustrates rheology and bead test in microgel composition of comparative polymer kept at pH 4.5 and 5.5 for 3 months at 45° C.

FIG. 3 illustrates rheology and bead test of microgel composition prepared using acids (glycolic and salicylic acid) and kept at pH 4.0 and 5.0 for 3 months at 45° C.

FIG. 4 illustrates rheology and bead test of microgel composition prepared using cationic polymer (guar hydroxypropyl trimonium chloride) and kept at pH 4.5 and 5.5 for 3 months 45° C.

FIG. 5 illustrates rheology and bead test of a microgel composition prepared using surfactant (sodium alpha olefin sulfonate) and kept at pH 4.0 and 5.0 for 3 months at 45° C.

FIG. 6 illustrates rheology and bead test of microgel composition for series of polymer ranges.

FIG. 7 illustrates rheology and bead test of microgel composition for series of polymer ranges.

FIG. 8 illustrates rheology and bead test of microgel composition using scale-up batch of emulsion polymer prepared using acids (glycolic and salicylic acid) and kept at pH 4.0 and 5.0 for 3 months at 45° C.

FIG. 9 illustrates rheology and bead test of microgel composition using scale-up batch of polymer prepared using cationic polymer (guar hydroxypropyl trimonium chloride) and kept at pH 4.5 and 5.5 for 3 months at 45° C.

FIG. 10 illustrates a comparison study of rheology and bead test for polymer 7 and commercial polymer for 3 months at 45° C. for 1 week at pH 4.5, wherein the aqueous polymer 7 shows higher viscosity, higher yield and more stability. Commercial polymer 7 failed bead test and stability test at 3 months.

### DETAILED DESCRIPTION OF THE INVENTION

While this specification concludes with claims particularly pointing out and distinctly claiming that which is regarded as the invention, it is anticipated that the invention can be more readily understood through reading the following detailed description of the invention and study of the included examples.

Numerical ranges as used herein are intended to include every number and subset of numbers contained within that range, whether specifically disclosed or not. Further, these numerical ranges should be construed as providing support for a claim directed to any number or subset of numbers in that range.

All references to singular characteristics or limitations of the present invention shall include the corresponding plural characteristic or limitation, and vice-versa, unless otherwise specified or clearly implied to the contrary by the context in which the reference is made.

References herein to “one embodiment,” or “one aspect” or “one version” or “one objective” of the invention may include one or more of such embodiment, aspect, version or objective, unless the context clearly dictates otherwise.

All publications, articles, papers, patents, patent publications, and other references cited herein are hereby incorporated herein by reference for all purposes to the extent consistent with the disclosure herein.

As used herein, the term “alkali swellable or alkali soluble thickener” refers to carboxyl containing copolymers produced by the addition or free radical polymerization of ethylenically unsaturated monomers and that swell or solubilize to thicken aqueous media on neutralization are commonly referred to as alkali-swellable or alkali-soluble thickeners.

As used herein, the term “cationic polymer” as used herein, indicates any polymer containing cationic groups and/or ionizable groups in cationic groups. The suitable cationic polymers are chosen from among those containing units including primary, secondary, tertiary, and/or quaternary amine groups.

As used herein, the term “comprising” refers to various optional, compatible components that can be used in the compositions herein, provided that the important ingredients are present in the suitable form and concentrations. The term “comprising” thus encompasses and includes the more restrictive terms “consisting of” and “consisting essentially of” which can be used to characterize the essential ingredients of the disclosed composition.

As used herein, the term “ethyl acrylate free” refers to the presence of ethyl acrylate as monomer or residual impurity in not more than [NMT] 1 mole % of the total polymer composition.

As used herein, the term “polymer” refers to a compound comprising repeating structural units (monomers) connected by covalent chemical bonds. The definition includes oligomers. Polymers can be further derivatized (example by hydrolysis), crosslinked, grafted or end-capped. Non-limiting examples of polymers include copolymers, terpolymers, quaternary polymers, and homologues. A polymer can be a random, block, or an alternating polymer, or a polymer with a mixed random, block, and/or alternating structure. Polymers can further be associated with solvent adducts.

As used herein, the term “polymeric rheology modifier” refers to a polymer system which provides various rheological properties, such as flow properties, thickening or viscosity, vertical cling, suspending ability, and yield value. It can be measured using a number of techniques, such as via the use of a constant stress rheometer or via extrapolation using a Brookfield viscometer.

As used herein, the term “surfactant” refers to a chemical compound which, when dissolved in water, concentrate at surfaces (interfaces) such as water-air or water-oil depending on their molecular structure giving rise to wide range of surface chemistry functions such as wetting, emulsifying, solubilising, foaming/defoaming, rheology modifying, anti-static, glossing, lubricity and surface conditioning.

As used herein, the term “thickener” refers to the relative increase in viscosity or thickening effect produced by the addition of a minimum amount of polymer of the present application as compared to the viscosity produced by a same amount of another polymer or thickening agent.

As used herein, the words “preferred,” “preferably” and variants thereof refer to embodiments of the invention that afford certain benefits, under certain circumstances. However, other embodiments can also be preferred, under the same or other circumstances. Furthermore, the recitation of one or more preferred embodiments does not imply that other embodiments are not useful, and is not intended to exclude other embodiments from the scope of the invention.

As used herein, the abbreviation “qs” refers to quantity sufficient.

As used herein, the term “Yield Value” refers to the initial resistance to flow under stress or minimum shear stress required to induce flow. The unit used to define this parameter is Pa or Pascal.

As used herein, the term “Zero shear viscosity” refers to the Viscosity [shear stress/shear rate] of a composition at rest. The symbol and units used for Viscosity are q and Pa·s [Pascal·second] respectively.

What is described herein is an aqueous emulsion polymer, which swells in the presence of at least one surfactant to form a swellable microgel, a process for preparing the same and the use of the swellable microgel as a rheology modifying agent in home care, industrial care and personal care applications.

In one embodiment, the present application provides an aqueous emulsion polymer comprising (a) monomers: (i) from about 12 mole % to about 27 mole % of butyl acrylate, (ii) from about 23 mole % to about 50 mole % of methyl acrylate; and (iii) from about 30 mole % to about 50 mole % of methacrylic acid; (b) from about 200 ppm to about 3000 ppm of at least one crosslinking agent; and (c) from about 0.5 wt. % to about 2.0 wt. % of at least one anionic or non-ionic surfactant.

According to one embodiment, ethyl acrylate is present in an amount not more than [NMT] 1 mole % of the total polymer. The applicants have found that ethyl acrylate having safety and regulatory issues can be effectively replaced by other alternative acrylic monomers such as butyl acrylate and methyl acrylate which surprisingly showed an enhanced viscosity shifting the polymer composition to a lower pH as compared to polymers having crosslinked or uncross linked ethyl acrylate or methacrylic acid monomers.

Hence the present application provides an aqueous emulsion polymer formed by emulsion polymerisation of monomers butyl acrylate, methyl acrylate and methacrylic acid reacted in the presence of a cross-linking agent and at least one surfactant. The emulsion polymer is substantially free of ethyl acrylate or even if present, it will be not more than (NMT) 1 mole % of the polymer composition. The representative chemical structures of the monomers employed for preparing the emulsion polymer are provided below:

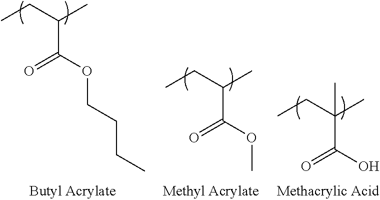

The crosslinking agent is selected from the group consisting of, but not limited to, allyl ethers of sucrose or of pentaerythritol, or similar compounds, diallyl esters, dimethallyl ethers, allyl or methallyl acrylates and acrylamides, tetraallyl tin, tetra vinyl silane, polyalkenyl methanes, diacrylates and dimethacrylates, divinyl compounds such as divinyl benzene, divinyl glycol, polyallyl phosphate, diallyloxy compounds, phosphite esters, and the like. Typical of such polyunsaturated monomers are di, tri, or tetra, penta, or hexa-allyl sucrose; di, tri, or tetra-allyl pentaerythritol; diallylphthalate, diallyl itaconate, diallyl fumarate, diallyl maleate, divinylbenzene, allyl methacrylate, allyl citrate, ethylene glycol di(meth)acrylate, trimethylolpropane triacrylate, 1,6-hexanediol diacrylate, pentaerythritol triacrylate, tetramethylene diethacrylate, tetramethylene diacrylate, ethylene diacrylate, ethylene dimethacrylate, triethylene glycol methacrylate, and methylene bisacrylamide.

Specific, examples of cross-linking agents include, but are not limited to, divinyl ethers of an aliphatic diol of 1,2-ethanediol, 1,4-propanediol, 1,4-butnaediol, 1,5-pentanediol, 1,6-hexanediol, 1,7-heptanediol, 1,8-octanediol, 1,9-nonanediol, 1,10-decanediol, 1,11-undecanediol, 1,12-dodecendiol, divinyl ethers of diethylene glycol of triethylene glycol, tetraethylene glycol, pentaethylene glycol; hexaethylene glycol, heptaethylene glycol, octaethylene glycol, nonaethylene glycol, decaethylene glycol; polyalkylene glycols and acrylates thereof of polyethylene glycol diacrylate, trimethylolpropane triacrylate, propylene glycol diacrylate, polyhydric alcohols esterified once or twice with acrylic acid triallylamine, tetraallylethylenediamine, diallyl phthalate, pentaerythritol triallyl ether, pentaerythritol triacrylate, pentaerythritol tetraacrylate, N,N′-divinylimidazolidone, triallyl-1,3,5-triazine-2,4,6(1H,3H,5H)-trione and 2,4,6-triallyloxy-1,3,5-triazine. The preferred cross-linking agent is pentaerythritol triallyl ether (PETE). The crosslinking agent is present in amount of about 200 ppm to about 3000 ppm based on the weight of monomer solids.

Surfactants employed to prepare emulsion polymer of this application are anionic or nonionic surfactants or mixtures thereof that are known in the art of aqueous surfactant compositions. Nonionic surfactants can be broadly defined as compounds containing a hydrophobic moiety and a nonionic hydrophilic moiety. Examples of the hydrophobic moiety can be alkyl, alkyl aromatic, dialkyl siloxane, polyoxyalkylene, and fluoro-substituted alkyls. Examples of hydrophilic moieties are polyoxyalkylene, phosphine oxides, sulfoxides, amine oxides, and amides. Suitable non-ionic surfactants include but are not limited to aliphatic (C₆-C₁₈) primary or secondary linear or branched chain acids, alcohols or phenols, alkyl ethoxylates, alkyl phenol alkoxylates (especially ethoxylates and mixed ethoxy/propoxy), block alkylene oxide condensate of alkyl phenols, alkylene oxide condensates of alkanols, ethylene oxide/propylene oxide block copolymers, semi-polar nonionics (e.g., amine oxides and phospine oxides), as well as alkyl amine oxides. Other suitable nonionics include mono or di alkyl alkanolamides and alkyl polysaccharides, sorbitan fatty acid esters, polyoxyethylene sorbitan fatty acid esters, polyoxyethylene sorbitol esters, polyoxyethylene acids, and polyoxyethylene alcohols.

Examples of suitable nonionic surfactants include coco mono or diethanolamide, coco diglucoside, alkyl polyglucoside, cocamidopropyl and lauramine oxide, polysorbate 20, ethoxylated linear alcohols, cetearyl alcohol, lanolin alcohol, stearic acid, glyceryl stearate, PEG-100 stearate, and oleth 20.

The anionic surfactant can be any anionic surfactant that is known in the art of aqueous surfactant compositions. Suitable anionic surfactants include but are not limited to alkyl sulfates, alkyl ether sulfates, alkaryl sulfonates, alkyl succinates, alkyl sulfosuccinates, N-alkoyl sarcosinates, alkyl phosphates, alkyl ether phosphates, alkyl ether carboxylates, alkylamino acids, alkyl peptides, alkoyl taurates, carboxylic acids, acyl and alkyl glutamates, alkyl isethionates, and α-olefin sulfonates, especially their sodium, potassium, magnesium, ammonium and mono, di and triethanolamine salts or mixtures thereof. The alkyl groups generally contain from 8 to 18 carbon atoms and can be unsaturated. The alkyl ether sulfates, alkyl ether phosphates and alkyl ether carboxylates can contain from 1 to 10 ethylene oxide or propylene oxide units per molecule, and preferably contain 1 to 3 ethylene oxide units per molecule.

Examples of suitable anionic surfactants include sodium and ammonium lauryl ether sulfate (with 1, 2, and 3 moles of ethylene oxide), sodium, ammonium, and triethanolamine lauryl sulfate, disodium laureth sulfosuccinate, sodium cocoyl isethionate, sodium C₁₂₋₁₄ olefin sulfonate, sodium laureth-6 carboxylate, sodium C₁₂₋₁₅ pareth sulfate, sodium methyl cocoyl taurate, sodium dodecylbenzene sulfonate, sodium cocoyl sarcosinate, triethanolamine monolauryl phosphate, and fatty acid soaps.

In another specific embodiment, the present application provides an aqueous emulsion polymer comprising: (a) monomers: (i) about 20 to about 25 mole % of butyl acrylate, (ii) about 40 to about 45 mole % of methyl acrylate and (iii) about 33 to about 40 mole % of methacrylic acid; (b) about 1000 ppm of crosslinking agent pentaerythritol triallyl ether; and 1 wt. % of surfactant which is represented in the following reaction:

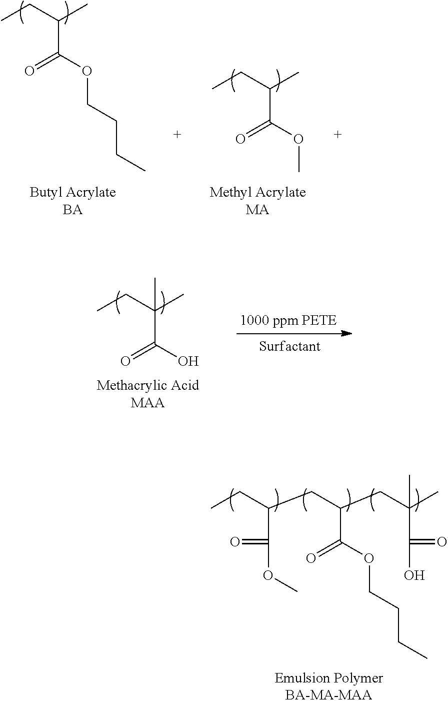

In another embodiment, the present application provides an aqueous microgel composition comprising: (i) an aqueous emulsion polymer of: (a) monomers: (i) from about 12 mole % to about 27 mole % of butyl acrylate; (ii) from about 23 mole % to about 50 mole % of methyl acrylate; and (iii) from about 30 mole % to about 50 mole % of methacrylic acid; (b) from about 200 ppm to about 3000 ppm of at least one cross-linking agent; and (c) from about 0.5 wt. % to about 2.0 wt. % of at least one anionic or non-ionic surfactant; (ii) at least one anionic, cationic, non-ionic or amphoteric surfactant or mixtures thereof; and (iii) optionally at least one acid, at least one cationic polymer, at least one oil alone or in combinations thereof.

The surfactants employed in the present application include cationic, anionic, non-ionic, amphoteric, or zwitterionic, mixtures thereof. These surfactants can be any surfactants that are known or previously used in the art of aqueous surfactant compositions. Zwitterionic surfactants are exemplified by those which can be broadly described as derivative of aliphatic quaternary ammonium, phosphonium, and sulfonium compounds in which the aliphatic radicals can be straight or branched chain, and wherein one of the aliphatic substituents contains from about 8 to about 18 carbon atoms and one contains as anionic water-solubilizing group, e.g., carboxy, sulfonate, sulfate, phosphate, or phosphonate. Examples of amphoteric surfactants which can be used in the vehicle systems of the cleansing composition of the present application are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate.

Suitable amphoteric or zwitterionc surfactants include, but are not limited to, alkyl betaines, alkyl amidopropyl betaines, alkyl sulphobetaines, alkyl glycinates, alkyl carboxy glycinates, alkyl amphopropionates, alkyl amidopropyl hydroxysultaines, acyl taurates and acyl glutamates wherein the alkyl and acyl groups have from 8 to 18 carbon atoms and mixtures thereof. Examples include cocamidopropyl betaine, sodium cocoamphoacetate, cocamidopropyl hydroxysultaine, and sodium cocamphopropionate. Amphoteric or zwitterionic surfactants can be used alone in combinations thereof.

Nonionic surfactants can be broadly defined as compounds containing a hydrophobic moiety and a nonionic hydrophilic moiety. Examples of the hydrophobic moiety can be alkyl, alkyl aromatic, dialkyl siloxane, polyoxyalkylene, and fluoro-substituted alkyls. Examples of hydrophilic moieties are polyoxyalkylene, phosphine oxides, sulfoxides, amine oxides, and amides. Suitable non-ionic surfactants include, but are not limited to, aliphatic (C₆-C₁₈) primary or secondary linear or branched chain acids, alcohols or phenols, alkyl ethoxylates, alkyl phenol alkoxylates (especially ethoxylates and mixed ethoxy/propoxy), block alkylene oxide condensate of alkyl phenols, alkylene oxide condensates of alkanols, ethylene oxide/propylene oxide block copolymers, semi-polar nonionics (e.g., amine oxides and phospine oxides), as well as alkyl amine oxides. Other suitable nonionics include mono or di alkyl alkanolamides and alkyl polysaccharides, sorbitan fatty acid esters, polyoxyethylene sorbitan fatty acid esters, polyoxyethylene sorbitol esters, polyoxyethylene acids, and polyoxyethylene alcohols.

Examples of suitable nonionic surfactants include coco mono or diethanolamide, coco diglucoside, alkyl polyglucoside, cocamidopropyl and lauramine oxide, polysorbate 20, ethoxylated linear alcohols, cetearyl alcohol, lanolin alcohol, stearic acid, glyceryl stearate, PEG-100 stearate, and oleth 20.

The anionic surfactant can be any anionic surfactant that is known in the art of aqueous surfactant compositions. Suitable anionic surfactants include, but are not limited to, alkyl sulfates, alkyl ether sulfates, alkaryl sulfonates, alkyl succinates, alkyl sulfosuccinates, N-alkoyl sarcosinates, alkyl phosphates, alkyl ether phosphates, alkyl ether carboxylates, alkylamino acids, alkyl peptides, alkoyl taurates, carboxylic acids, acyl and alkyl glutamates, alkyl isethionates, and α-olefin sulfonates, especially their sodium, potassium, magnesium, ammonium and mono, di and triethanolamine salts or mixtures thereof. The alkyl groups generally contain from 8 to 18 carbon atoms and can be unsaturated. The alkyl ether sulfates, alkyl ether phosphates and alkyl ether carboxylates can contain from 1 to 10 ethylene oxide or propylene oxide units per molecule, and preferably contain 1 to 3 ethylene oxide units per molecule.

Examples of suitable anionic surfactants include sodium and ammonium lauryl ether sulfate (with 1, 2, and 3 moles of ethylene oxide), sodium, ammonium, and triethanolamine lauryl sulfate, disodium laureth sulfosuccinate, sodium cocoyl isethionate, sodium C₁₂-14 olefin sulfonate, sodium laureth-6 carboxylate, sodium C₁₂-15 pareth sulfate, sodium methyl cocoyl taurate, sodium dodecylbenzene sulfonate, sodium cocoyl sarcosinate, triethanolamine monolauryl phosphate, and fatty acid soaps.

The cationic surfactants can be any of the cationic surfactants known or previously used in the art of aqueous surfactant compositions. Suitable cationic surfactants include, but are not limited to, alkyl amines, alkyl imidazolines, ethoxylated amines, quaternary compounds, and quaternized esters. In addition, alkyl amine oxides can behave as a cationic surfactant at a low pH. Examples include lauramine oxide, dicetyldimonium chloride, and cetrimonium chloride. Cationic surfactants can be used alone in combinations thereof.

While the amount of the above surfactants (anionic, cationic, non-ionic, amphoteric or zwitterionic) can vary widely, amounts which are often utilized generally in the present application range from about 0.5% to about 80%, from about 5% to about 65%, from about 6% to about 30% or from about 0.5% to 2.0%, or from about 1.0% to about 1.5% by weight based upon the total weight of the composition.

Cationic polymers employed in the present emulsion microgel composition can be any polymers that are known or previously used in the art of aqueous surfactant compositions. Cationic polymers used in the present application can be selected from, but not limited to, polyamine, polyaminoamide, and quaternary polyammonium types of polymers, such as: (1) homopolymers and copolymers derived from acrylic or methacrylic esters or amides. The copolymers can contain one or more units derived from acrylamides, methacrylamides, diacetone acrylamides, acrylamides and methacrylamides, acrylic or methacrylic acids or their esters. Specific examples include, but not limited to, copolymers of acrylamide and dimethyl amino ethyl methacrylate quaternized with dimethyl sulfate or with an alkyl halide; copolymers of acrylamide and methacryloyl oxyethyl trimethyl ammonium chloride; the copolymer of acrylamide and methacryloyl oxyethyl trimethyl ammonium methosulfate; (2) derivatives of cellulose ethers containing quaternary ammonium groups, such as hydroxy ethyl cellulose quaternary ammonium that has reacted with an epoxide substituted by a trimethyl ammonium group; (3) derivatives of cationic cellulose such as cellulose copolymers or derivatives of cellulose grafted with a hydro soluble quaternary ammonium monomer, as described in U.S. Pat. No. 4,131,576, such as the hydroxy alkyl cellulose, and the hydroxymethyl, hydroxyethyl or hydroxypropyl cellulose grafted with a salt of methacryloyl ethyl trimethyl ammonium, methacrylamidopropyl trimethyl ammonium, or dimethyl diallyl ammonium; (4) cationic polysaccharides such as described in U.S. Pat. Nos. 3,589,578 and 4,031,307, guar gums containing cationic trialkyl ammonium groups such as guar hydroxypropyl trimonium chloride, guar gums modified by a salt, e.g., chloride of 2,3-epoxy propyl trimethyl ammonium, Cassia, Chitosan and Chitin; (5) polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers; (6) water-soluble polyamino amides prepared by polycondensation of an acid compound with a polyamine. These polyamino amides can be reticulated; (7) derivatives of polyamino amides resulting from the condensation of polyalcoylene polyamines with polycarboxylic acids followed by alcoylation by bifunctional agents; (8) polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon; (9) the cyclopolymers of alkyl diallyl amine or dialkyl diallyl ammonium such as the homopolymer of dimethyl diallyl ammonium chloride and copolymers of diallyl dimethyl ammonium chloride and acrylamide; (10) quaternary diammonium polymers such as hexadimethrine chloride; (11) quaternary polyammonium polymers, including, for example, Mirapol® A 15, Mirapol® AD1, Mirapol® AZ1, and Mirapol® 175 products sold by Miranol; (12) Quaternary polyamines; and (13) Reticulated polymers known in the art.

Suitable cationic hetero polymer can be selected from the group consisting of, but not limited to, compounds having α,β-ethylenically unsaturated double bond and at least one cationogenic and/or cationic group per molecule; esters of α,β-ethylenically unsaturated mono and dicarboxylic acids with amino alcohols, C₂-C₂₀ amino alcohols which are C₁-C₈ mono or dialkylated on the nitrogen atom of the amine functional group having components of esters selected from acrylic acid, methacrylic acid, fumaric acid, maleic acid, itaconic acid, crotonic acid, maleic anhydride, monobutyl maleate alone or in combination thereof. Acrylic acid, methacrylic acid and mixtures thereof are particularly useful. The cationic heteropolymer can be selected from a group of quaternized ammonium compounds such as diethyldiallyl ammonium chloride (DEDAAC) dimethyldiallyl ammonium chloride (DMDAAC), methacryloyloxy ethyl trimethyl ammonium methylsulfate (METAMS), methacrylamido propyl trimethyl ammonium chloride (MAPTAC), acryloyloxyethyl trimethyl ammonium chloride (AETAC), methacryloyloxyethyl trimethyl ammonium chloride (METAC), acrylamidomethylpropyl trimethyl ammonium chloride (AMPTAC), acrylamido methyl butyl trimethyl ammonium chloride (AMBTAC) and mixtures thereof. Particularly useful cationic-containing monomers are MAPTAC, DMDAAC, DEDAAC and METAC alone or copolymerized with acrylamide, methacrylamide and N,N-dimethylacrylamide, alone or in combination.

Non-limiting examples of cationic polymers for the present application include, but are not limited to, N-tert-butyl amino ethyl(meth) acrylate, N,N-dimethyl aminomethyl (meth)acrylate, N,N-dimethyl amino ethyl (meth)acrylate, N,N-diethyl amino ethyl (meth)acrylate, N,N-dimethyl aminopropyl (meth)acrylate, N,N-diethyl amino propyl (meth)acrylate and N,N-dimethyl amino cyclohexyl (meth)acrylate, dimethyl aminomethyl acrylate, diethyl amino methyl acrylate, dimethyl amino ethyl acrylate, dimethyl amino butyl acrylate, dimethyl amino butyl methacrylate, dimethyl amino amyl methacrylate, diethyl amino amyl methacrylate, dimethyl amino hexyl acrylate, diethyl amino hexyl methacrylate, dimethyl amino octyl acrylate, dimethyl amino octyl methacrylate, diethyl amino octyl acrylate, diethyl amino octyl methacrylate, dimethyl amino decyl methacrylate, dimethyl amino dodecyl methacrylate, diethyl amino lauryl acrylate, diethyl amino lauryl methacrylate, dimethyl amino stearyl acrylate, dimethyl amino stearyl methacrylate, diethyl amino stearyl acrylate and diethyl amino stearyl methacrylate. Particularly useful are N-tert-butylaminoethyl(meth)acrylate and N,N-dimethylaminoethyl(meth)acrylate. Particular preference is furthermore given to N,N-dimethylaminoethyl acrylate and N,N-dimethylaminoethyl methacrylate. Further, the suitable amide based cationic non-homopolymer can be selected from a group of compounds including, but not limited to, α,β-ethylenically unsaturated mono and dicarboxylic acids with diamines having at least one primary or secondary amino group in it. The choice is provided to diamines which have one tertiary and one primary or secondary amino group. The most appropriate monomers would include, but are not limited to, N-tert-butyl amino ethyl (meth)acrylamide, N-[2-(dimethylamino) ethyl]acrylamide, N-[2-(dimethylamino) ethyl] methacrylamide, N-[3-(dimethylamino) propyl]acrylamide, N-[3-(dimethylamino) propyl] methacrylamide, N-[4-(dimethylamino) butyl]acrylamide, N-[4-(dimethylamino) butyl] methacrylamide, N-[2-(diethylamino) ethyl]acrylamide, N-[4-(dimethylamino) cyclohexyl] acrylamide and N-[4-(dimethylamino) cyclohexyl] methacrylamide, N-[12-(dimethylamino) dodecyl]-methacrylamide, N-[18-(dimethylamino) octadecyl] methacrylamide, N-[8-(dimethylamino) octyl] methacrylamide, N-[7-(dimethylamino) heptyl] acrylamide, N-[14-(dimethylamino) tetradecyl] acrylamide, N-[3-(dimethylamino) propyl] methacrylamide, N-[3-(diethylamino) propyl] acrylamide, N-(4-(dipropylamino) butyl] methacrylamide, N-[3-(methyl butyl amino) propyl] acrylamide, N-(2-[3-(dimethylamino) propyl]ethyl) acrylamide, N-(4-[4-(diethylamino) butyl]butyl)acrylamide. Special significance is given to N-[3-(dimethylamino)propyl] acrylamide, N-[3-(dimethylamino)propyl] methacrylamide (DMAPMA) and mixtures thereof.

According to another embodiment of the present application, one or more various cationic polymers belonging to the “polyquaternium” (PQ) family of polymers can be employed to prepare the emulsion polymer. The suitable PQ compounds include, but are not limited to: PQ-2, PQ-4, PQ-5, PQ-6, PQ-7, PQ-8, PQ-9, PQ-10, PQ-11, PQ-14, PQ-16, PQ-17, PQ-18, PQ-19, PQ-20, PQ-21, PQ-22, PQ-24, PQ-27, PQ-28, PQ-29, PQ-31, PQ-32, PQ-37, PQ-39, PQ 41, PQ-42, PQ-44, PQ-46, PQ-47, PQ-48, PQ-49, PQ-50, PQ-55, PQ-67, PQ-69, PQ-77 and other quaternary ammonium compounds are listed in the CTFA Cosmetic Ingredient Handbook, First Edition, on pages 41-42, incorporated herein by reference, and are described in the “History of Polymers in Hair care,” Cosmetics and Toiletries, 103 (1988), incorporated herein by reference. Other natural, semi-natural and synthetic polymers that can be used with the present application can be referenced in the CTFA Dictionary, Fifth Edition, 2000, incorporated herein by reference.

Examples of cationic polymers that can be use in the proactive of this invention include water-soluble polymers such as anionic, hydrophobically-modified, and amphoteric acrylic acid copolymers, vinylpyrrolidone homopolymers; cationic, hydrophobically-modified, and amphoteric vinylpyrrolidone copolymers; nonionic, cationic, anionic, and amphoteric cellulosic polymers such as hydroxyethylcellulose, hydroxypropylcellulose, carboxymethylcellulose, hydroxypropylmethylcellulose, cationic hydroxyethylcellulose, cationic carboxymethylhydroxyethylcellulose, and cationic hydroxypropylcellulose; acrylamide homopolymers and cationic, amphoteric, and hydrophobically-modified acrylamide copolymers, polyethylene glycol polymers and copolymers, hydrophobically-modified polyethers, hydrophobically-modified polyetheracetals, hydrophobically-modified polyols and polyetherurethanes and other polymers referred to as associative polymers, hydrophobically-modified cellulosic polymers, polyethyleneoxide-propylene oxide copolymers, and nonionic, anionic, hydrophobically-modified, amphoteric, and cationic polysaccharides such as xanthan, chitosan, carboxymethyl guar, alginates, hydroxypropyl guar, carboxymethyl guar hydroxypropyl trimethyl ammonium chloride, guar hydroxypropyl trimethyl ammonium chloride, cationic cassia polymer, hydroxypropyl guar hydroxypropyl trimethyl ammonium chloride.

While the amount of cationic polymer can vary widely, the suitable amounts which are often utilized generally range from about 0.05% to about 10%, from about 0.5% to about 5%, by weight based upon the total weight of the composition.

Acids used in the present application are any cosmetically acceptable acid, salt or ester which does not eliminate the functional benefit of the compound (e.g., its hydrating, nourishing, or metabolic enhancing properties). Acid is monoacid, diacid or mixtures thereof. Examples of cosmetically acceptable acids, esters, salts, or mixtures thereof include, but are not limited to organic acids (e.g., acetic, lactic, maleic, citric, maleic, ascorbic, succinic, benzoic, methane sulfonic, toluenesulfonic, or palmoic acid), as well as polymeric acids (e.g., tannic or carboxymethyl cellulose) and salts with inorganic acids such as a hydrohalic acid (e.g., hydrochloric acid, sulfuric acid, or phosphoric acid). Examples of cosmetically acceptable esters include, but are not limited to C₂-C₆ alkyl esters such as methyl esters and ethyl esters. Examples include but are not limited to creatine monohydrate, creatine hemisulfate, D-carnitine, L-carnitine, L-carnitine hydrochloride, sodium pyruvate, and pyruvic acid methyl ester. As used herein, if the stereochemistry of the compound is not indicated then the compound includes all stereoisomers, if any. Preferred acids can be selected from alpha-hydroxy acid, beta-hydroxy acid (salicylic acid), glycolic acid and citric acid.

Oil used in the application includes, but is not limited to, organic argon oil, olive squalene, olive oil, castor oil, coconut oil, palm oil, soybean oil, cottonseed oil, sweet almond oil, avocado oil, grape seed oil, jojoba oil, tea tree oil, meadow foam seed oil, macadamia nut oil, wheat germ oil or oil with a known cosmetic, functional or therapeutic benefit.

The prior art polymeric rheology modifiers do not provide substantial viscosity until a pH of at least about 6 or 7 is achieved. There are some home and personal care applications where there is a need for pH of less than 6 for demonstrating optimal and desired performance. Additionally, it is difficult to even formulate stable end use applications having polymers at this lower pH range. Such compositions are increased to a near neutral or even alkaline pH and then subsequently reduced in pH, the viscosity and yield value generally remain unchanged or increase. The desired pH to stabilize compositions of the present application is obviously dependent on the specific end use applications. Generally, personal care applications have a desired pH range of about 3 to about 7.5, desirably from about 4 to about 6. Generally, home care applications have a desired pH range of about 1 to about 12, and desirably from about 3 to about 10. More specifically, when a generally insoluble silicone or pearlescent compound is utilized, a desired pH is from about 5.5 to about 12, whereas when a hair dye is stabilized, the pH is from about 5 to about 9. Skin care customers are shifting surfactant based formulations to a lower pH such as for use in face washes which contain salicylic acid to treat acne. Currently, there is no ideal solution available on the market.

The present aqueous emulsion polymers thicken at pH below 7.0. They are activated by the presence of surfactants (cationic, anionic, nonionic, amphoteric, zwitterionic or mixtures thereof) to form isotropically clear to opaque microgels as needed in aqueous media primarily used to suspend all types of beads, chemical peels, air bubbles, oil droplets, and exfoliating agents.

As a result, the yield, viscosity, suspension and stabilization of the compositions are enhanced through a direct pH adjustment in the presence of acids, cationic polymers and surfactants. Emulsion polymer is used as a rheology modifier and suspending aid for surfactant based micelle technology, emulsions and complex fluids.

In yet another embodiment, the present application provides use of stable aqueous emulsion polymer as rheology modifier compositions in personal care applications, home care applications, industrial and institutional applications, and the like.

In non-structured liquid detergent or personal care products, the presence of such ingredients generally leads to sedimentation or phase separation and therefore renders such products unacceptable from a consumer's viewpoint.

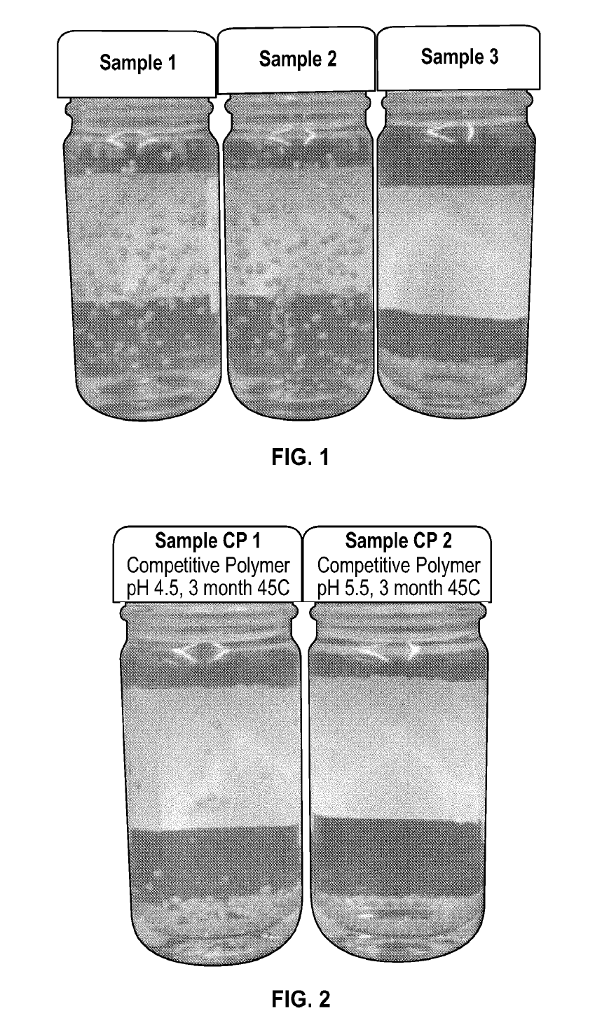

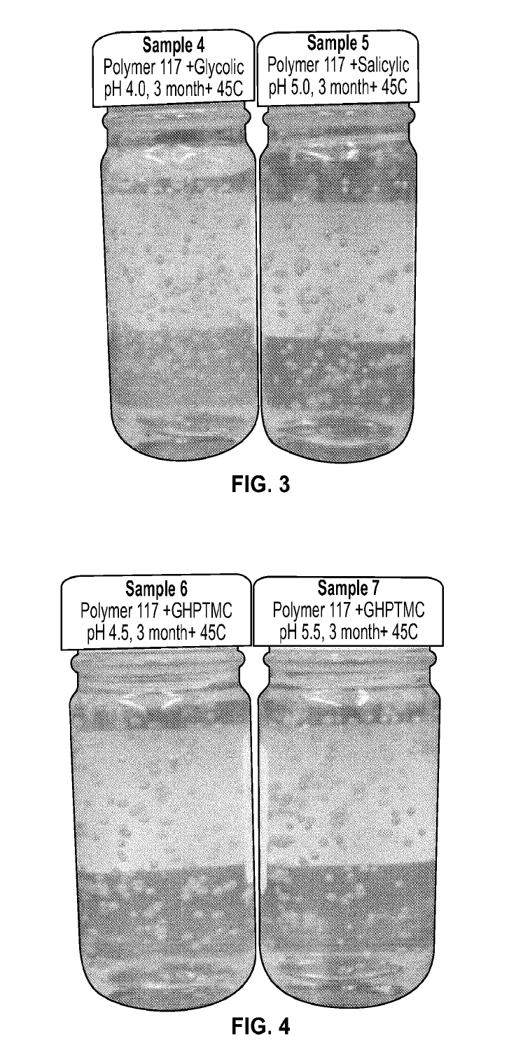

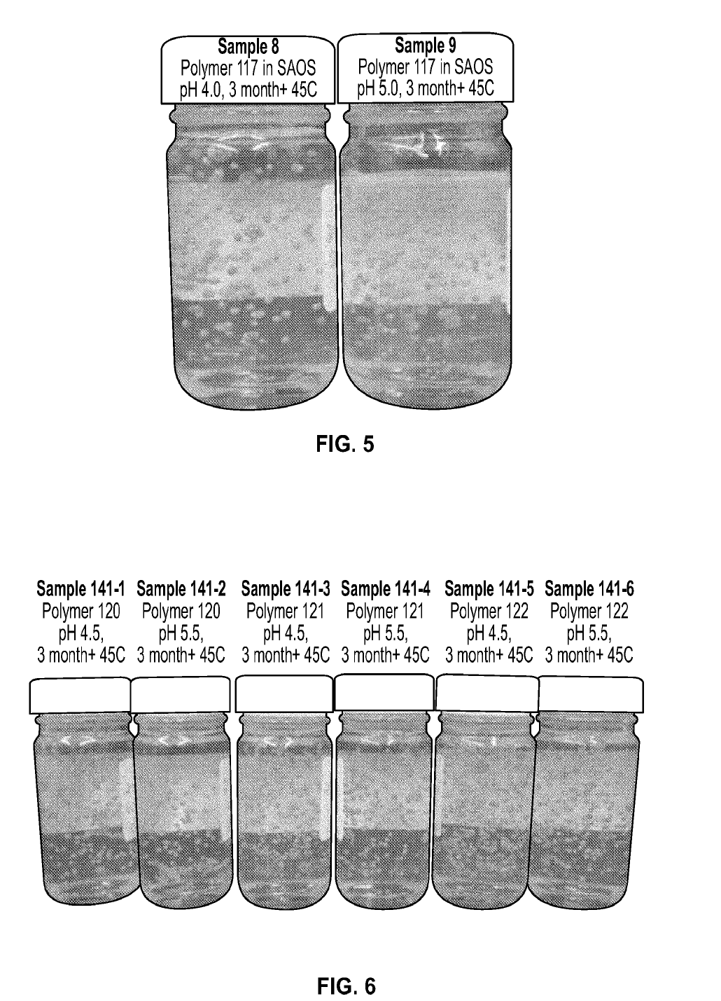

The aqueous emulsion polymer was used for preparing the microgel optionally using cosmetically acceptable acids and cationic polymer. These different compositions when subjected to rheology studies and stability tests showed improved rheology. FIG. 1 illustrates rheology and bead test in microgel composition kept at pH 4.5, 5.5 and 6.5 for 3 month at 45° C., wherein Yield Value and Zero shear viscosity were found to be 3.5 Pa, 24,870 Pa·s; 2.5 pa, 24260 Pa·s and 0.6 Pa and 973 Pa·s respectively. Under similar conditions, commercial polymer was tested which showed low Yield Value and Zero shear viscosity 0.33 Pa, 1218 Pa·s and 0.27 Pa·s, 779 Pa·s respectively, results shown in FIG. 2. FIG. 3 illustrates rheology and bead test of microgel composition prepared using acids (glycolic and salicylic acid) and kept at pH 4.0 and 5.0; for 3 months at 45° C., wherein Yield Value and Zero shear viscosity were found to be 4.7 Pa, 33210 Pa·s and 2.6, 28960 Pa·s respectively. When microgel compositions comprising emulsion polymer and cationic polymer kept at pH 4.5, 5.5; for 3 months at 45° C. were tested for rheology performance, improved yield value and zero shear viscosity were observed 3.7 Pa, 31440 Pa·s and 2.6 Pa, 30100 Pa·s respectively. FIG. 5 illustrates rheology and bead test of microgel composition prepared using surfactant (sodium alpha olefin sulfonate) and kept at pH 4.0 and 5.0 for 3 months at 45° C. wherein the results show yield value and zero shear viscosity to be 3.7 Pa, 40650 Pa·s and 1.9 Pa, 23190 Pa·s respectively.

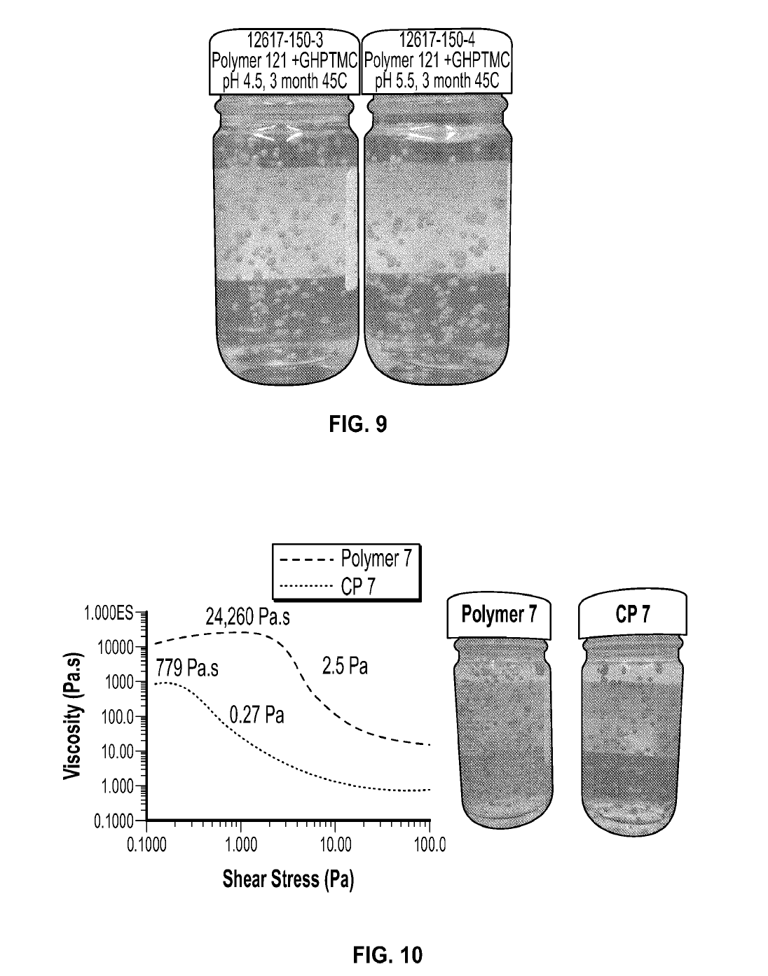

The present microgel was found to be stable even on scale-up batch thereby producing consistent results of viscosity and yield value. The results remain the same even in the presence of acids or cationic polymers as shown in FIG. 8: 4.3 Pa, 28440 Pa·s and 1.9 Pa, 17550 Pa·s respectively; and FIG. 9: 2.9 Pa, 21190 Pa·s and 1.9 Pa, 15600 Pa·s respectively. The present microgels are compatible with different types of acids, cationic polymers thereby making them effective for end use formulations to retain the suspension hold and rheology.

The present application optionally includes additives for home care applications not limited to laundry detergents, dishwashing detergents (automatic and manual), hard surface cleaners, hand soaps, cleaners and sanitizers, polishes (shoe, furniture, metal, etc.), automotive waxes, polishes, protectants, and cleaners, and the like.

In yet another embodiment, the present application provides an aqueous microgel based personal care composition comprising: (i) an aqueous emulsion polymer of: (a) monomers: (i) from about 12 mole % to about 27 mole % of butyl acrylate; (ii) from about 23 mole % to about 50 mole % of methyl acrylate; and (iii) from about 30 mole % to about 50 mole % of methacrylic acid; (b) from about 200 wt. ppm to about 3000 wt. ppm of at least one cross-linking agent; and (c) from about 0.5 wt. % to about 2.0 wt % of at least one anionic or non-ionic surfactant; (ii) at least one anionic, cationic, non-ionic, zwitterionic or amphoteric surfactant or mixtures thereof; (iii) at least one personal care additive; and (iv) optionally at least one acid, at least one cationic polymer, at least one oil alone or combinations thereof.

Personal care compositions comprising polymer(s) according to the present application include sun care compositions, after-sun compositions, hair care compositions, conditioning compositions, skin care compositions, oral care compositions, face care compositions, lip care compositions, eye care compositions, body care compositions, nail care compositions, foot care, anti-aging compositions, insect repellants, deodorant compositions, color cosmetic compositions, color protection compositions, and self-tanning compositions.

Suitable personal care or cosmetically acceptable ingredients or conventional additives that are currently employed are well known in the relevant art and can be readily chosen by an artisan including, but not limited to, hair styling agents, auxiliary fixatives and film formers that modify the hair attributes such as gums, resins, polymers of synthetic or natural origin, and the like; chemical hair waving or straightening agents; hair colorants such as pigments and dyes for temporary, semi-permanent, or permanent hair dyeing; polymer film modifying agents plasticizers, tackifiers, detackifiers, wetting agents and the like, product finishing agents, structurants, gelling agents, chelating agents, proteins, amino acids, vitamins and/or provitamins, opacifiers, pearlescing agents, resins, preservatives, fragrances, solubilizers, butter, penetrants, colorants pigments and dyes; UV absorbers, and the like; propellants (water miscible or water immiscible) such as fluorinated hydrocarbons, liquid volatile hydrocarbons, compressed gases, other additives such as volatiles, liquid vehicles, carriers, salts, anti-frizz agents, anti-dandruff agents, relaxers, fatty substances, hydrophilic or lipophilic active agent, reducing agents, thickeners, electrolytes, pH adjusting agents, organosilicon compounds, anti-foaming agents, penetrants, hair and skin conditioning agents such as antistatic agents, synthetic oils, vegetable or animal oils, silicone oils, monomeric or polymeric quaternized ammonium salts, emollients, humectants, lubricants, sunscreen agents, and the like, chelating agents, antimicrobial agents, anti-oxidants, anti-radical protecting agents, natural extracts, lubricants, combing aids or coalescing agents, solubilizers, neutralizing agents, vapor pressure suppressants, bleaching agents, hydrating agents, moisturizers, protectives (for example, antiradical agents), abrasives, emulsifiers (including, but not limited to ethoxylated fatty acids, ethoxylated glyceryl esters, ethoxylated oils, ethoxylated sorbitan esters, fatty esters, PEG esters, polyglycerol esters), antiperspirants (including, but not limited to aluminium chlorohydrates, aluminium zirconium chlorhydrates), botanicals, cleansing agents, associative polymers, oils of vegetable, mineral, and/or synthetic origin, polyols, silicones, colorants, bleaching agents, highlighting agents, styling polymers, benefit agents, skin lighteners (including, but not limited to arbutin and kojic acids), tanning agents (including, but not limited to dihydroxyacetone) and combinations thereof.

The suitable non-limiting moisturizers/humectants employed in the present application include glycols, glycerols, propylene glycol, diethylene glycol monoethyl ether, sorbitol, sodium salt of pyroglutamic acid, glycerol, glycerol derivatives, glycerin, trehalose, sorbitol, maltitol, dipropylene glycol, 1,3-butylene glycol, sodium hyaluronate, and the like.

The functional silicones are selected from the group consisting of silicone quat, silicone fluid and silicone wax. These silicones are able to decrease friction during hair combing and flat ironing, smooth gliding; increase conditioning and smoothing to hair by adding flexibility; help reducing heat damage and style retention and contribute to shine depending on the refractive index.

The reducing agents are typically selected from thioglycolate, thiosulfate, sulfite, bisulfite, and urea. These agents target disulfide bonds to create additional disulfide interchange and rearrangement.

The oxidizing agents are typically selected from hydrogen peroxide, bromides and peracetic acid to fix the disulfide bond back.

The conditioning agents are typically selected from polymeric and small quaternary ammonium compounds such as tertiary amines quaternium and polyquaternium compounds.

The humectants are typically selected from glycols, sugars, oils, silicones, emollients and proteins.

The pH adjusting buffers can be selected from the group consisting of, but not limited to, glyoxylic acid, maleic acids, succinic acid, formic acid and other organic and inorganic acids. These buffers promote active penetration of the composition of the present application and facilitate the reactions if any for effective results of the composition. Non-limiting examples of acidifying or acidic pH adjusting agents include organic acids, such as citric acid, acetic acid, carboxylic acids, α-hydroxyacids, β-hydroxyacids, α,β-hydroxyacids, salicylic acid, tartaric acid, lactic acid, glycolic acid, natural fruit acids, and combinations thereof. In addition, inorganic acids, for example hydrochloric acid, nitric acid, sulfuric acid, sulfamic acid, phosphoric acid, and combinations thereof can be utilized.

Non-limiting examples of alkalizing or alkaline pH adjusting agents include ammonia, alkali metal hydroxides (such as sodium hydroxide and potassium hydroxide), ammonium hydroxide, alkanolamines (such as mono-, di- and triethanolamine), diisopropylamine, dodecylamine, diisopropanolamine, aminomethyl propanol, cocamine, oleamine, morpholine, triamylamine, triethylamine, tromethamine (2-amino-2-hydroxymethyl)-1,3-propanediol), and tetrakis(hydroxypropyl)ethylenediamine, hydroxyalkylamines and ethoxylated and/or propoxylated ethylenediamines, alkali metal salts of inorganic acids, such as sodium borate (borax), sodium phosphate, sodium pyrophosphate, and the like, and mixtures thereof.

Suitable buffering agents include, but are not limited to, alkali or alkali earth carbonates, phosphates, bicarbonates, citrates, borates, acetates, acid anhydrides, succinates and the like, such as sodium phosphate, citrate, borate, acetate, bicarbonate, and carbonate.

The preservatives include different classes of compounds such as organic acids (sorbic, salicylic, dehydroacetic, benzoic, 4-hydroxybenzoic, etc.), alkyl esters of benzoic acid, alkyl esters of alkyl-p-hydroxybenzoic acids (parabens methyl paraben, ethyl paraben, propyl paraben, butyl paraben, and benzyl paraben), phenol derivatives (o-phenyl-phenol, 4-chloro-m-cresol, etc.), carbanilides (triclocarban). Specific examples include, but not limited to, propylene glycol, phenoxyethanol, caprylyl glycol, iodopropynyl butyl carbamate, Optiphen MIT Ultra and diazolidinyl urea.

The compositions optionally contain other rheology modifiers to be used in conjunction with the aqueous emulsion polymer of this application. These polymers are well known in the art and can include natural, semi-synthetic (e.g. clays), or synthetic polymers. Examples of natural or modified natural polymers include but are not limited to gums (e.g., xanthan gum), cellulosics, modified cellulosics, starches, or polysaccharides. Examples of other synthetic polymers include but are not limited to cross-linked polyacrylates, hydrophobically modified alkali-soluble polymers, or hydrophobically modified nonionic urethane polymers. Additionally, the adjustment of viscosity by admixture of salt is also well known and can be employed in the present application. If present in a composition, these rheology modifiers are generally used from about 0.01 to about 5% by weight of the stable composition.

Suitable pearlescent agents can be selected from the group consisting of, but not limited to, alkylene glycol esters, ethylene glycol distearate; fatty acid alkanolamides, coco fatty acid diethanolamide; partial glycerides, especially stearic acid monoglyceride; esters of polybasic, optionally hydroxy substituted carboxylic acids with fatty alcohols containing 6 to 22 carbon atoms, especially long-chain esters of tartaric acid; fatty compounds, for example fatty alcohols, fatty ketones, fatty aldehydes, fatty ethers and fatty carbonates which contain in all at least 24 carbon atoms, laurone and distearylether; fatty acids, such as stearic acid, hydroxy stearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof. Other pearlizing agents include titanium dioxide, zinc oxide, mica, polystyrene derivatives.

In yet another embodiment, the present application provides a process for preparing an aqueous emulsion polymer, the process comprising: (a) preparing a pre-emulsion by mixing solutions of monomers: (i) from about 12 mole % to about 27 mole % of butyl acrylate; (ii) from about 23 mole % to about 50 mole % of methyl acrylate; and (iii) about 30 mole % to about 50 mole % of methacrylic acid; about 200 ppm to about 3000 ppm of at least one cross-linking agent; and about 0.5 wt. % to about 2.0 wt. % of at least one anionic or non-ionic surfactant in the dispersion medium; (b) adding polymerisation initiator and monomer pre-emulsion of step (a) to a reactor; (c) polymerizing reactants of step (b) at 75-80° C. for about 3-6 hours; and (d) cooling the reaction solution to obtain the aqueous emulsion polymer.

Non-limiting examples of free radical initiators include water soluble inorganic persulfate compounds, such as ammonium persulfate, potassium persulfate, and sodium persulfate; inorganic sulfate compounds such as ferrous ammonium sulfate; peroxides, such as hydrogen peroxide, benzoyl peroxide, acetyl peroxide, and lauryl peroxide; organic hydroperoxide, such as cumene hydroperoxide and t-butyl hydroperoxide; organic per acids, such as peracetic acid and perbenzoic acid (optionally activated with reducing agents, such as sodium bisulfite or ascorbic acid); and oil soluble, free radical producing agents, such as 2,2′-azobisisobutyronitrile, and the like.

The swellable microgel comprising aqueous emulsion polymer provides desired yield, viscosity, suspension and stabilization through a direct pH adjustment in the presence of acids, cationic polymers and surfactants. The flow rheology is also optimized for surfactant based systems. These properties are achieved at pH below 7.0.

Further, certain aspects of the present application are illustrated in detail by way of the following examples. The examples are given herein for illustration of certain aspects of the present application and are not intended to be limiting thereof.

### EXAMPLES

### Example 1: Emulsion Polymer

Pre-Emulsion Preparations:

Reactor charge was prepared using surfactant sodium laureth sulfate (5.5 g) and deionised water (234 g). Initiator shot was prepared using ammonium persulphate (0.126 g) and deionised water (2 g). Monomer charge included methyl acrylate (79.89 g), butyl acrylate (64 g), methacrylic acid (66.15 g), and crosslinker pentaerythritol triallyl ether (70%) (0.3 g). Initiator feed was prepared using ammonium persulfate (0.084 g) and deionised water (29.92 g). Aqueous phase was prepared with deionised water (210 g) and sodium laureth sulfate (1.83 g). The monomer phase and aqueous phase homogenized together followed by continuous stirring throughout the reaction using magnetic flea and magnetic stirrer plate to form pre-emulsion monomer feed.

Post-Emulsion Preparations:

Ammonium Iron (II) sulphate hexahydrate (FAS) and water were combined to produce a 0.05% active solution. The 0.05% FAS solution was weighed to produce the promoter solution shot. Ascorbic acid (0.069 g) and deionised water (1.5 g) were combined to produce two reductant solution shots. 70% active tert-butyl hydroperoxide (tBHP) (0.051 g) was weighed to produce two oxidant shots.

Reaction Procedure:

Water (234 g) was charged into a reactor vessel maintained at 80° C. in a water bath. Surfactant sodium laureth sulfate (5.5 g) from the reactor charge was added into the reactor vessel and stirred until homogenous. The monomer feed and initiator shot prepared were added to the reactor vessel via calibrated metered pump for about 90 and 120 minutes respectively. A promoter solution was added to the reactor vessel as a single shot. The polymerisation was held for 5 minutes. The residual monomers were reduced using a redox initiator system for a further 2 hours. The reaction was cooled to room temperature and a preservative sorbic acid (0.7 g) was added to isolate the polymer product.

The reaction was then cooled to room temperature by submerging the reactor vessel in a cold water bath.

### Example 2: Microgel Composition Preparation Using Acid—Standard Process

An emulsion polymer obtained in example 1 was added to deionized water in a main beaker. Anionic surfactant was added later and employed propeller mixing at moderate speed to ensure uniformity. Betaine was added followed by propeller mixing and pH was measured. Acid such as glycolic acid or salicylic acid was added and mixing was continued until the acid is completely dissolved and uniformly dispersed. The pH was measured and adjusted accordingly either by adding citric acid or sodium hydroxide to retain the desired pH. Formulation was allowed to stand overnight and finally pH, clarity and viscosity were measured.

### Example 3: Microgel Composition Preparation Using Cationic Polymer—Standard Process

An emulsion polymer obtained in example 1 was added to deionized water in a main beaker. Anionic surfactant was added later and employed propeller mixing at moderate speed to ensure uniformity. Betaine was added followed by propeller mixing and pH was measured. Cationic polymer is added as a prehydrated solution with continued propeller mixing until uniformly dispersed. The pH was measured and adjusted accordingly either by adding citric acid or sodium hydroxide to reach the desired pH. Formulation was allowed to stand overnight and finally pH, clarity and viscosity were measured.

### Example 4: Rheology Study

Three similar compositions were tested for stability at different pH 4.51 (Sample 1); pH 5.47 (Sample 2) and pH 6.47 (Sample 3) and illustrated in FIG. 1. It was observed that composition remained stable even at pH 6.47. The solution is clear with improved viscosity and yield which determines the suspension factor.

| | Sample 1 | Sample 2 | Sample 3 |
|---|---|---|---|
| Ingredient Name | (%) | (%) | (%) |
| Deionized Water | 43.33 | 43.33 | 43.33 |
| Polymer + (30%) | 6.67 | 6.67 | 6.67 |
| preservative (0.1%) | | | |
| Sodium Laureth | 40.00 | 40.00 | 40.00 |
| Sulfate (25%) | | | |
| Cocamidopropyl | 10.00 | 10.00 | 10.00 |
| Betaine (30%) | | | |
| Citric Acid (25% | qs to pH 4.5 | qs to pH | qs to pH |
| Solution) | | 5.5 | 6.5 |
| Starting PH | 5.4 | 5.33 | 5.35 |
| Adjusted PH | 4.51 | 5.47 | 6.47 |
| Final PH | 4.51 | 5.47 | 6.47 |
| Appearance | very slight | Very slight | Clear |
| | hazy | hazy | |
| Haze (NTU) | 46.4 | 43.7 | 23.8 |
| Viscosity TC @ 10 | 46400 | 22700 | 13300 |

### Example 5: Microgel Composition Comparison Study Using Comparative Polymer (CP)

Comparative polymers were tested for stability at pH 4.5 and 5.5. After two weeks beads went down and no improved yield for commercial polymer. The results are illustrated in FIG. 2.

| | Commercial | Commercial |
|---|---|---|
| Ingredient Name | Sample 1 | Sample 2 |
| Deionized Water | 43.33 | 43.33 |
| CP (30%) | 6.67 | 6.67 |
| Sodium Laureth | 40.00 | 40.00 |
| Sulfate (25%) | | |
| Cocamidopropyl | 10.00 | 10.00 |
| Betaine | | |
| Citric Acid (25% | qs to pH 4.5 | qs to pH 5.5 |
| Solution) | | |
| Starting PH | 5.23 | 5.22 |
| Adjusted PH | 4.48 | 5.49 |
| Final PH | 4.48 | 5.49 |
| Appearance | Clear | Clear |
| Haze (NTU) | 31.3 | 28.6 |
| Viscosity TC @ 10 | 4300 | 3100 |

### Example 6: Microgel Composition Comparison Study Using Polymers Prepared Incorporating Ethyl Acrylate

Microgels were prepared using emulsion polymers prepared using the method from example 1 containing ethyl acrylate (samples A and B). Samples were tested at pH 4.5. Samples showed decreased viscosity and yield compared to sample 1 in Example 4. Under these conditions EA monomer free compositions showed novel, unique, behavior in providing suspension and viscosity.

| Ingredient Name | Sample A | Sample B |
|---|---|---|
| Deionized Water | 43.33 | 43.33 |
| EA/MAA Polymer 1 (30%) | 6.67 | — |
| EA/MAA Polymer 2 (30%) | — | 6.67 |
| Sodium Laureth Sulfate | 40.00 | 40.00 |
| (25%) | | |
| Cocamidopropyl Betaine | 10.00 | 10.00 |
| (30%) | | |
| Citric Acid (25% Solution) | qs to pH | qs to pH |
| | 4.5 | 4.5 |
| Starting pH | 5.77 | 5.73 |
| Adjusted pH | 4.49 | 4.44 |
| Final pH | 4.61 | 4.5 |
| Appearance | *Slt Hazy | Hazy |
| Haze (NTU) | 45.2 | 61.9 |
| Viscosity TC @ 10 | 16500 | 9600 |

*Slt: Slight

### Example 7: Rheology Study on Microgel Composition with Acids

The application emulsion polymer forms strong gel in acids. Hence the emulsion polymer is compatible to different types of acids unlike commercial polymer. Viscosity and yield stress improved. The results are illustrated in FIG. 3.

| Ingredient Name | Sample 4 (%) | Sample 5 (%) |
|---|---|---|
| Deionized Water | 36.19 | 41.33 |
| Polymer 3188-117 | 6.67 | 6.67 |
| (30%) | | |
| Sodium Laureth Sulfate | 40.00 | 40.00 |
| (25%) | | |
| Cocamidopropyl Betaine | 10.00 | 10.00 |
| (30%) | | |
| Glycolic Acid (70%) | 7.14 | |
| Salicylic Acid (100%) | | 2.00 |
| pH before Acid | 5.25 | 5.25 |
| pH after Acid | 2.86 | 3.66 |
| Adjust pH to 4.0 | 4.00 | 5.00 |
| Final pH | 3.96 | 5.00 |
| Haze (NTU) | 106 | 56 |
| Viscosity TC@ 10 | >100000 | 20500 |

### Example 8: Rheology Study Using Microgel Composition with Cationic Polymer

Microgel has cationic polymer and emulsion polymer has the ability to attract anionic substrates such as skin and hair, thereby compatible to different finished products. The results are illustrated in FIG. 4.

| Ingredient Name | Sample 6 (%) | Sample 7 (%) |
|---|---|---|
| Deionized Water | 33.33 | 33.33 |
| Polymer 3188-117 | 6.67 | 6.67 |
| (30%) | | |
| Sodium Laureth | 40 | 40 |
| Sulfate (25%) | | |
| Cocamidopropyl | 10 | 10 |
| Betaine (30%) | | |
| Guar hydroxypropyl | 10 | 10 |
| trimonium chloride | | |
| (2%) | | |
| pH Before cationic | 5.26 | 5.29 |
| polymer | | |
| pH after cationic | 5.31 | 5.36 |
| polymer | | |
| Citric Acid (25% | QS to 4.5 | QS to 5.5 |
| Solution) | | |
| Final pH | 4.52 | 5.52 |
| Haze (NTU) | 61.7 | 55.5 |
| Viscosity TC @10 | 52400 | 25100 |

### Example 9: Rheology Study on Microgel Composition Using Surfactant Sodium α-Olefin Sulfonate

Studies were conducted using different surfactant sodium α-olefin sulfonate replacing sodium laureth sulfonate as used in previous examples. Microgel was found to be stable at pH 5.0 for 3 months at 45° C. The results are illustrated in FIG. 5.

| Ingredient Name | Sample 8 (%) | Sample 9 (%) |
|---|---|---|
| Phase A | | |
| Deionized Water | 45.53 | 45.53 |
| Glycerin | 3.00 | 3.00 |
| Polymer 3188-117 | 6.67 | 6.67 |
| (30%) | | |
| Sodium Alpha | 30.00 | 30.00 |
| Olefin Sulfonate | | |
| (40%) | | |
| Phase B | | |
| Salicylic Acid | 2.00 | 2.00 |
| Phase C | | |
| Optiphen MIT | 0.30 | 0.30 |
| Ultra | | |
| Phase D | | |
| C12-15 Alkyl | 0.50 | 0.50 |
| Lactate | | |
| Phase E | | |
| Cocamidopropyl | 12.00 | 12.00 |
| Betaine (30%) | | |
| Adjust pH to | 4.00 | 5.00 |
| pH | 4.02 | 5.02 |
| Haze (NTU) | 35.6 | 48.0 |
| Viscosity TC@10 | 39700 | 13800 |

### Example 10: Microgel Composition Rheology Testing a Design of Experiment (DOE) with Crosslinked Emulsion Polymers

Rheology studies were performed on various polymers (labeled 120, 121, 122) with varied ranges and different crosslinkers. The results are illustrated in FIG. 6. It was observed that the bead suspension was found to be stable even at 5.5 pH for 3 months at 45° C.

| | Sample | Sample | Sample | Sample | Sample | Sample |
|---|---|---|---|---|---|---|
| | 141-1 | 141-2 | 141-3 | 141-4 | 141-5 | 141-6 |
| Ingredient Name | (%) | (%) | (%) | (%) | (%) | (%) |
| Deionized Water | 42.33 | 42.33 | 42.33 | 42.33 | 42.33 | 42.33 |
| Polymer 120 (30%) | 6.67 | 6.67 | — | — | — | — |
| Polymer 121 (30%) | — | — | 6.67 | 6.67 | — | — |
| Polymer 122 (30%) | — | — | — | — | 6.67 | 6.67 |
| Polymer 123 (30%) | — | — | — | — | — | — |
| Polymer 124 (30%) | — | — | — | — | — | — |
| Polymer 125 (30%) | — | — | — | — | — | — |
| Sodium Laureth | 40.00 | 40.00 | 40.00 | 40.00 | 40.00 | 40.00 |
| Sulfate (25%) | | | | | | |
| Cocamidopropyl | 10.00 | 10.00 | 10.00 | 10.00 | 10.00 | 10.00 |
| Betaine (25%) | | | | | | |
| Diazolidinyl Urea | 1.00 | 1.00 | 1.00 | 1.00 | 1.00 | 1.00 |
| and Methyl paraben | | | | | | |
| and Propyl paraben | | | | | | |
| and Propylene Glycol | | | | | | |
| Citric Acid | qs to | qs to | qs to | qs to | qs to | qs to |
| (25% Solution) | pH 4.5 | pH 5.5 | pH 4.5 | pH 5.5 | pH 4.5 | pH 5.5 |
| Starting PH | 5.34 | 5.37 | 5.36 | 5.38 | 5.34 | 5.38 |
| Adjusted PH | 4.48 | 5.47 | 4.50 | 5.47 | 4.47 | 5.48 |
| Final PH | 4.48 | 5.47 | 4.50 | 5.47 | 4.47 | 5.48 |
| Haze (NTU) | 35.3 | 27.5 | 42.3 | 36.3 | 44.6 | 36.8 |
| Viscosity TC @10 | 49000 | 25500 | 56900 | 26600 | 57300 | 28600 |

### Example 11: Microgel Composition Testing a Design of Experiment (DOE) with Crosslinked Polymers 123, 124 and 125

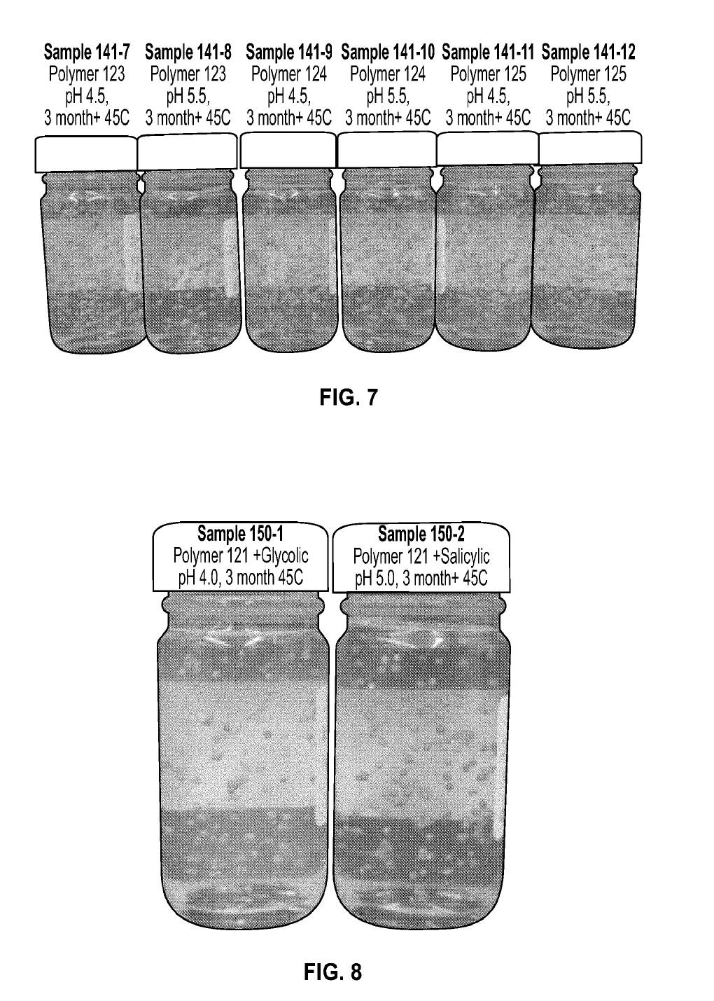

Rheology studies were performed on various polymers (labeled 123, 124, 125) with varied ranges and different crosslinkers. The results are illustrated in FIG. 7. It was observed that the bead suspension was found to be stable even at 5.5 pH for 3 months at 45° C.

| | Sample | Sample | Sample | Sample | Sample | Sample |
|---|---|---|---|---|---|---|
| | 141-7 | 141-8 | 141-9 | 141-10 | 141-11 | 141-12 |
| Ingredient Name | (%) | (%) | (%) | (%) | (%) | (%) |
| Deionized Water | 42.33 | 42.33 | 42.33 | 42.33 | 42.33 | 42.33 |
| Polymer 123 (30%) | 6.67 | 6.67 | — | — | — | — |
| Polymer 124 (30%) | — | — | 6.67 | 6.67 | — | — |
| Polymer 125 (30%) | — | — | — | — | 6.67 | 6.67 |
| Sodium Laureth | 40.00 | 40.00 | 40.00 | 40.00 | 40.00 | 40.00 |
| Sulfate (25%) | | | | | | |
| Cocamidopropyl | 10.00 | 10.00 | 10.00 | 10.00 | 10.00 | 10.00 |
| Betaine (30%) | | | | | | |
| Diazolidinyl Urea | 1.00 | 1.00 | 1.00 | 1.00 | 1.00 | 1.00 |
| and Methyl paraben | | | | | | |
| and Propyl paraben | | | | | | |
| and Propylene Glycol | | | | | | |
| Citric Acid | qs to | qs to | qs to | qs to | qs to | qs to |
| (25% Solution) | pH 4.5 | pH 5.5 | pH 4.5 | pH 5.5 | pH 4.5 | pH 5.5 |
| Starting PH | 5.34 | 5.32 | 5.36 | 5.35 | 5.38 | 5.37 |
| Adjusted PH | 4.5 | 5.49 | 4.48 | 5.47 | 4.5 | 5.48 |
| Final PH | 4.5 | 5.49 | 4.48 | 5.47 | 4.5 | 5.48 |
| Haze (NTU) | 52.3 | 46.2 | 61.9 | 47.9 | 69.5 | 59.9 |
| Viscosity TC @ 10 | 56600 | 28100 | 49200 | 29400 | 57600 | 28800 |

### Example 12: Rheology Study on Microgel Composition with Acids

Rheology studies were performed on microgel emulsion polymer samples prepared scale-up batch. Glycolic acid and salicylic acid were added in different samples and it was observed that bead suspension was intact and stable. On scale-up batch, amount of cross linker required to bring about the same rheology was reduced. Results are illustrated in FIG. 8.

| Ingredient | | |
|---|---|---|
| Name | Sample 150-1 (%) | Sample 150-2 (%) |
| Deionized Water | 36.19 | 41.33 |
| Polymer 3188- | 6.67 | 6.67 |
| 121 (30%) | | |
| Sodium Laureth | 40.00 | 40.00 |
| Sulfate (25%) | | |
| Cocamidopropyl | 10.00 | 10.00 |
| Betaine (30%) | | |
| Glycolic Acid | 7.14 | |
| (70%) | | |
| Salicylic Acid | | 2.00 |
| (100%) | | |
| pH before Acid | 5.30 | 5.29 |
| pH after Acid | 2.86 | 3.35 |
| Adjust pH to 4.0 | 4.00 | 5.00 |
| glyc/5.0 sal. | | |
| Final pH | 3.97 | 5.02 |
| Haze (NTU) | | 44.2 |
| Viscosity TC@ | 28440 | 17550 |
| 10 | | |

### Example 13: Rheology Study on Microgel Composition with Cationic Polymer

Rheology studies were performed on microgel emulsion polymer samples prepared on pilot scale. Cationic polymer guar hydroxy propyl trimethyl ammonium chloride was added in different samples and it was observed that bead suspension was intact and stable. On pilot scale amount of cross linker required to bring about the same rheology was reduced. Results are illustrated in FIG. 9.

| Ingredient Name | Sample 150-3 (%) | Sample 150-4 (%) |
|---|---|---|
| Deionized Water | 33.33 | 33.33 |
| Polymer 3188-121 | 6.67 | 6.67 |
| (30%) | | |
| Sodium Laureth | 40.00 | 40.00 |
| Sulfate (25%) | | |
| Cocamidopropyl | 10.00 | 10.00 |
| Betaine (30%) | | |
| Guar | 10.00 | 10.00 |
| hydroxypropyl | | |
| trimonium | | |
| Chloride | | |
| (GHPTMC) (2%) | | |
| pH Before | 5.26 | 5.24 |
| GHPTMC | | |
| pH after | 5.30 | 5.27 |
| GHPTMC | | |
| Citric Acid (25% | QS to 4.5 | QS to 5.5 |
| Solution) | | |
| Final PH | 4.50 | 5.51 |
| Haze (NTU) | | 47.6 |
| Viscosity TC @10 | 68800 | 26700 |

### Example 14: Facial Cleanser Formulation with Emulsion Polymer Microgel

A detailed personal care application of microgel is provided below.

Phase A is a polymer (3188-117) (5-7 wt. %) along with surfactant sodium alpha olefin sulfonate (35 wt. %) and water (43.03 wt. %) were mixed in a beaker. Phase B has salicylic acid (2.0 wt. %), glycerin (3.0 wt. %), C₁₂-C₁₅ alkyl lactate (0.25 wt. %), preservative mixture phenoxyethanol and caprylyl glycol (1.0 wt. %), fragrance (0.25 wt. %) were pre-mixed to ensure acid is thoroughly dispersed. Contents of phase B were mixed to phase A contents in beaker. Phase C is cationic polymer cocamidopropyl betaine (8.77 wt. %) which was mixed to beaker containing phase A and phase B and mixing was continued for at least 45 minutes followed by measurement of pH (3.77). pH was later adjusted to 4.0. Phase D has captivates GL 7184 (1.0 wt. %), FD&C Red 40 (0.20 wt. %), and FD&C yellow 6 (0.5 wt. %) were mixed to above solution. Final pH was measured (4.19) to obtain the facial cleansing solution.

While this invention has been described in detail with reference to certain preferred embodiments, it should be appreciated that the present invention is not limited to those precise embodiments. Rather, in view of the present disclosure, many modifications and variations would present themselves to those skilled in the art without departing from the scope and spirit of this invention.

What is claimed is:

1. An aqueous emulsion polymer comprising:
a) monomers: i) from about 12 mole % to about 27 mole % of butyl acrylate; ii) from about 23 mole % to about 50 mole % of methyl acrylate; and iii) from about 30 mole % to about 50 mole % of methacrylic acid;
b) from about 200 ppm to about 3000 ppm of at least one crosslinking agent; and
c) from about 0.5 wt. % to 2.0 wt. % of at least one anionic or non-ionic surfactant.

2. The aqueous emulsion polymer according to claim 1, wherein said ethylacrylate monomer is present in not more than 1.0 mole % of the total polymer composition.

3. The aqueous emulsion polymer according to claim 1, wherein said crosslinking agent is selected from the group consisting of divinyl ethers of an aliphatic diol of 1,2-ethanediol, 1,3-propanediol, 1,4-butanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol, divinyl ethers of diethylene glycol of triethylene glycol, tetraethylene glycol, pentaethylene glycol; hexaethylene glycol, heptaethylene glycol, octaethylene glycol, nonaethylene glycol, decaethylene glycol, polyalkylene glycols and acrylates of polyethylene glycol diacrylate, trimethylolpropane triacrylate, propylene glycol diacrylate, polyhydric alcohols esterified with acrylic acid triallylamine, tetraallylethylenediamine, diallyl phthalate, pentaerythritol triallyl ether, pentaerythritol triacrylate, pentaerythritol tetra-acrylate, N,N′-divinylimidazolidone, triallyl-1,3,5-triazine-2,4,6(1H,3H,5H)-trione and 2,4,6-triallyloxy-1,3,5-triazine alone or in combinations thereof.

4. The aqueous emulsion polymer according to claim 1, wherein said at least one non-ionic surfactant is selected from the group consisting of aliphatic (C₆-C₁₈) primary, secondary, linear or branched chain acids, alcohols or phenols, alkyl ethoxylates, alkyl phenol alkoxylates, block alkylene oxide condensate of alkyl phenols, alkylene oxide condensates of alkanols, ethylene oxide or propylene oxide block copolymers, semi-polar nonionics amine oxides or phosphine oxides, mono or di alkyl alkanolamides, alkyl polysaccharides, sorbitan fatty acid esters, polyoxyethylene sorbitan fatty acid esters, polyoxyethylene sorbitol esters, polyoxyethylene acids, polyoxyethylene alcohols, coco mono or diethanolamide, coco diglucoside, alkyl polyglucoside, cocamidopropyl and lauramine oxide, polysorbate 20, ethoxylated linear alcohols, cetearyl alcohol, lanolin alcohol, stearic acid, glyceryl stearate, PEG-100 stearate, oleth 20 alone or in combinations thereof; at least one anionic surfactant selected from the group consisting of sodium and ammonium lauryl ether sulfate having 1, 2, or 3 ethylene oxide units; sodium, ammonium, and triethanol amine lauryl sulfate, disodium laureth sulfosuccinate, sodium cocoyl isethionate, sodium C₁₂₋₁₄ olefin sulfonate, sodium laureth-6 carboxylate, sodium C₁₂₋₁₅ pareth sulfate, sodium methyl cocoyl taurate, sodium dodecylbenzene sulfonate, sodium cocoyl sarcosinate, and triethanolamine monolauryl phosphate alone or in combinations thereof.

5. The aqueous emulsion polymer according to claim 1,
wherein said butyl acrylate is present in an amount of about 20 to about 25 mole %, methyl acrylate is present in an amount of about 40 to about 45 mole %, methacrylic acid is present in an amount of about 33 to about 40 mole %;
wherein said crosslinking agent is present in an amount of about 1000 ppm; and
wherein said anionic or non-ionic surfactant is present in an amount of 1.0 wt. % of the total polymer composition.

6. An aqueous microgel composition comprising:
i. an aqueous emulsion polymer of: (a) monomers: (i) from about 12 mole % to about 27 mole % of butyl acrylate; (ii) from about 23 mole % to about 50 mole % of methyl acrylate; and (iii) from about 30 mole % to about 50 mole % of methacrylic acid; (b) from about 200 ppm to about 3000 ppm of at least one crosslinking agent; and (c) from about 0.5 wt. % to 2.0 wt. % of at least one anionic or non-ionic surfactant;
ii. at least one anionic, cationic, non-ionic, or amphoteric surfactant, or mixtures thereof; and
iii. optionally, at least one acid, at least one cationic polymer, or at least one oil alone or in combinations thereof.

7. The aqueous microgel composition according to claim 6, wherein said microgel is capable of thickening at pH 3 to 7 through direct pH adjustment.

8. The aqueous microgel composition according to claim 6, wherein said at least one non-ionic surfactant is selected from the group consisting of aliphatic (C₆-C₁₈) primary, secondary, linear or branched chain acids, alcohols or phenols, alkyl ethoxylates, alkyl phenol alkoxylates, block alkylene oxide condensate of alkyl phenols, alkylene oxide condensates of alkanols, ethylene oxide or propylene oxide block copolymers, semi-polar nonionics amine oxides or phosphine oxides, mono or di alkyl alkanolamides, alkyl polysaccharides, sorbitan fatty acid esters, polyoxyethylene sorbitan fatty acid esters, polyoxyethylene sorbitol esters, polyoxyethylene acids, polyoxyethylene alcohols, coco mono or diethanolamide, coco diglucoside, alkyl polyglucoside, cocamidopropyl and lauramine oxide, polysorbate 20, ethoxylated linear alcohols, cetearyl alcohol, lanolin alcohol, stearic acid, glyceryl stearate, PEG-100 stearate, oleth 20 alone or in combinations thereof; at least one anionic surfactant selected from sodium and ammonium lauryl ether sulfate having 1, 2, or 3 ethylene oxide units; sodium, ammonium, and triethanol amine lauryl sulfate, disodium laureth sulfosuccinate, sodium cocoyl isethionate, sodium C₁₂₋₁₄ olefin sulfonate, sodium laureth-6 carboxylate, sodium C₁₂₋₁₅ pareth sulfate, sodium methyl cocoyl taurate, sodium dodecylbenzene sulfonate, sodium cocoyl sarcosinate, and triethanolamine monolauryl phosphate alone or in combinations thereof; amphoteric surfactant is selected from the group consisting of alkyl betaines, alkyl amidopropyl betaines, alkyl sulphobetaines, alkyl glycinates, alkyl carboxy glycinates, alkyl amphopropionates, alkyl amidopropyl hydroxysultaines, acyl taurates and acyl glutamates wherein the alkyl and acyl groups have from 8 to 18 carbon atoms alone or in combinations thereof; cationic surfactant selected from the group consisting of alkyl amines, alkyl imidazolines, ethoxylated amines, quaternary compounds, and quaternized esters alone or in combinations thereof.

9. The aqueous microgel composition according to claim 6, wherein said acid is monoacid, diacid, or mixtures thereof selected from the group consisting of alpha-hydroxy acids; beta-hydroxy acids; organic acids acetic acid, lactic acid, maleic acid, citric acid, maleic acid, ascorbic acid, succinic acid, benzoic acid, methane sulfonic acid, toluenesulfonic acid, or palmoic acid; polymeric acids tannic acid; and salts with inorganic acids hydrohalic acid, sulfuric acid and phosphoric acid.

10. The aqueous microgel composition according to claim 6, wherein said cationic polymer is a homopolymer, copolymer or terpolymer selected from the group consisting of diethyldiallyl ammonium chloride (DEDAAC) dimethyldiallyl ammonium chloride (DMDAAC), methacryloyloxy ethyl trimethyl ammonium methylsulfate (METAMS), methacrylamido propyl trimethyl ammonium chloride (MAPTAC), acryloyloxyethyl trimethyl ammonium chloride (AETAC), methacryloyloxyethyl trimethyl ammonium chloride (METAC), acrylamidomethylpropyl trimethyl ammonium chloride (AMPTAC), acrylamido methyl butyl trimethyl ammonium chloride (AMBTAC) poly(acrylamido propyl trimethylammonium chloride) (polyAPTAC), poly(diallyl dimethyl ammonium chloride), poly(acrylamido propyl trimethyl ammonium chloride behenyl methacrylate acrylic acid) terpolymer, poly(acrylamido propyl trimethyl ammonium chloride stearyl acrylate acrylic acid) terpolymer, and poly(acrylamido propyl trimethyl ammonium chloride stearyl acrylate acrylamidopropyl methane sulfonic acid) terpolymer, nonionic, anionic, hydrophobically-modified, amphoteric, and cationic polysaccharides selected from xanthan, chitosan, cellulose, cassia, carboxymethyl guar, alginates, hydroxypropyl guar, carboxymethyl guar hydroxypropyltrimethylammonium chloride, guar hydroxypropyltrimethylammonium chloride, hydroxypropyl guar hydroxypropyltrimethylammonium chloride alone or in combinations thereof.

11. The aqueous microgel composition according to claim 6, wherein said aqueous emulsion polymer is used as rheology modifier and suspending aid for surfactant based micelle technology, emulsions and complex fluids.

12. The aqueous microgel composition according to claim 6, wherein said aqueous emulsion is used for preparing end-use applications including a personal care composition, a home care composition, an institutional care composition or an industrial care composition.

13. An aqueous personal care microgel composition comprising:
i. an aqueous emulsion polymer of: (a) monomers: (i) from about 12 mole % to about 27 mole % of butyl acrylate; (ii) from about 23 mole % to about 50 mole % of methyl acrylate; and (iii) from about 30 mole % to about 50 mole % of methacrylic acid; (b) from about 200 ppm to about 3000 ppm of at least one cross-linking agent; and (c) from about 0.5 wt. % to about 2.0 wt. % of at least one anionic or non-ionic surfactant;
ii. at least one anionic or cationic or non-ionic or amphoteric surfactant or mixtures thereof;
iii. at least one personal care additive; and
iv. optionally, at least one acid, at least one cationic polymer, at least one oil alone or in combinations thereof.

14. The aqueous personal care microgel composition according to claim 13, wherein the personal care additive is selected from the group consisting of preservatives, functional polymers, surfactants, propellants, perfumes, deodorants, anti-perspirant actives, sunscreen actives, hair treatment agents, oral care agents, laundry or detergent, fragrances, essential oils, jojoba oil, meadow foam water soluble silicones, natural or synthetic waxes, fatty acids and alcohols, conditioners, emollients, humectants, mineral oil and petrolatum, opacifying or pearlescent materials, particulates, auxiliary rheology modifiers, emulsifier, diluents, chelating agents, cleansing agents, fixatives, viscosifying agents, coloring agents alone or in combinations thereof.

15. A process for preparing an aqueous emulsion polymer comprising:
a) preparing a pre-emulsion by mixing solutions of monomers: (i) from about 12 mole % to about 27 mole % of butyl acrylate; (ii) from about 23 mole % to about 50 mole % of methyl acrylate; and (iii) about 30 mole % to about 50 mole % of methacrylic acid; about 200 ppm to about 3000 ppm of at least one cross-linking agent; and about 0.5 wt. % to about 2.0 wt. % of at least one anionic or non-ionic surfactant in a dispersion medium;
b) adding polymerisation initiator and monomer pre-emulsion of step (a) to a reactor;
c) polymerizing reactants of step (b) at 75-80° C. for about 3-6 hours; and
d) cooling the reaction solution to obtain the aqueous emulsion polymer.

16. The process according to claim 15, wherein said polymerisation initiator is selected from the group consisting of hydrogen peroxide, peracetic acid, t-butyl hydroperoxide, di-t-butyl peroxide, dibenzoyl peroxide, benzoyl hydroperoxide, 2,4-dichlorobenzoyl peroxide, 2,5-dimethyl-2,5-bis(hydroperoxy)hexane, perbenzoic acid, t-butyl peroxypivalate, t-butyl per acetate, dilauroyl peroxide, dicapryloyl peroxide, distearoyl peroxide, diisopropyl peroxydicarbonate, dodecyl peroxydicarbonate, dieicosyl peroxydicarbonate, di-t-butyl perbenzoate, azobisisobutyronitrile, 2,2′-azobis-2,4-dimethylvaleronitrile, ferrous ammonium sulfate, ammonium persulfate, potassium persulfate, sodium persulfate and sodium perphosphate alone or in combinations thereof.
